(19) United States
(12) Patent Application Publication
(10) Pub. No.: US 2026/0251651 A1
(43) Pub. Date: Aug. 27, 2026

(54) LATERAL FLOW ASSAY SYSTEMS AND METHODS FOR THE QUANTIFICATION OF A BIOLOGICAL SAMPLE

(71) Applicant: SANGUIS DIAGNOSTICS CORP., Loxahatchee, FL (US)

(72) Inventor: Patrick Lawless, Lexington, KY (US)

(21) Appl. No.: 19/649,549

(22) Filed: Apr. 16, 2026

Related U.S. Application Data
(63) Continuation of application No. 17/208,055, filed on Mar. 22, 2021.
Application No. 17/208,055 is a continuation of application No. PCT/US2020/057636, filed on Oct. 28, 2020.
(60) Provisional application No. 62/927,910, filed on Oct. 30, 2019.

(51) Int. Cl. G01N 33/543; G01N 33/53; G01N 33/74

(52) U.S. Cl. CPC G01N 33/54388; G01N 33/5302; G01N 33/54386; G01N 33/74; G01N 2333/62; G01N 2800/042

(57) ABSTRACT

A point of care testing method for equine insulin includes extracting an equine whole blood sample from a horse and placing the whole blood sample on a test strip of a lateral flow assay device. The test strip has a whole blood filter pad, a conjugate pad carrying a conjugated insulin detection antibody, and a nitrocellulose membrane carrying an insulin capture antibody that binds to the conjugated insulin detection antibody bound to the equine insulin from the conjugate pad and forms a visible test line on the test strip. An optoelectronic reader measures an intensity of the visible test line and the equine insulin concentration of the equine whole blood sample is displayed.

### CROSS REFERENCE TO RELATED APPLICATIONS

This is a continuation of application Ser. No. 17/208,055 filed Oct. 28, 2020, which is a continuation of PCT Application No. PCT/US2020/057636, filed Oct. 28, 2020, which claims the benefit of priority to Application No. 62/927,910, filed Oct. 30, 2019. All of the foregoing applications are incorporated herein by reference in their entirety.

### FIELD

The present disclosure relates in general to lateral flow assay systems, test devices, and methods.

### BACKGROUND

Lateral flow assays can provide reliable, inexpensive, portable, rapid, and simple diagnostic tests. However, traditionally designed lateral flow assays suffer from performance limitations, most notably low sensitivity and poor reproducibility. Lateral flow assays are routinely used to quantify one or more analytes that are present in test articles in the nanogram per milliliter range or higher. However, lateral flow assays are very rarely capable of reproducibly quantifying analytes present in test articles at concentrations less than 1 nanogram or 1,000 picograms per milliliter. Despite this performance limitation, there are many analytes, most notably hormones, that are present in test articles at low concentrations (1,000 picograms or less), which exert strong physiological effects, and therefore, are of particular interest to medical practitioners due to abnormal concentrations being indicative of health risks or disease states.

Whole blood is a preferable diagnostic test article in point of care settings because it can be easily and rapidly obtained without the labor and equipment required for serum and plasma sample preparation. Whole blood samples, however, contain endogenous substances that can adversely impact diagnostic assay performance through their interference with one or more components of the assay. Therefore, lateral flow assays using whole blood must be designed in such a manner that substances that could possibly interfere with the assay are taken into account as a “background” signal which can be subtracted from the true analyte signalment or by removal of interfering substances, for example via filtration through a blood filter pad or sample pad that selectively removes interfering substances but does not significantly affect the analyte in the sample matrix.

Therefore, there is a need for lateral flow assay diagnostic devices and methods that overcome the limitations of the current technologies and methodologies, which are less subject to interpretation errors, which can produce quantitative results in instances where analytes are present at low concentrations, that are reproducible, that can be multiplexed and that can be applied in point of care scenarios where whole blood may be the only test article available for rapid diagnosis.

### SUMMARY

In one aspect, a lateral flow assay test system is provided. The lateral flow assay test system comprises: a volumetric pipette; a chemical reagent solution referred to as a chase buffer or running buffer; a lateral flow assay test device; a test device housing including one or more ports; a reader, including a light source, a light detector, and a data analyzer.

In an embodiment, the lateral flow assay test device is configured to comprise a label and an agent configured to specifically bind to an analyte of interest.

In an embodiment, the lateral flow assay test device is configured to comprise a test strip that is further comprised of at least one of: a sample pad, a blood filter pad, a conjugate pad, a nitrocellulose membrane, a wick pad, an insulin antibody, a gold nanoparticle, and a detection agent.

In an embodiment, a port is an opening in the test device housing where a biological sample or a chemical reagent solution (“running buffer” or “chase buffer”) or a mixture thereof is applied to the test strip.

In an embodiment, the test strip is contained in a housing that is referred to as a cassette or cartridge.

In another aspect, a method of testing for a metabolic syndrome or disease in a horse is provided. The method comprises: obtaining a fluid sample from an equine mammal, mixing the fluid sample with the chemical reagent solution to form a testing sample, and contacting the biological fluid sample with a lateral flow assay test device.

In an embodiment, the lateral flow assay test device is capable of binding insulin in the biological fluid sample from an equine animal with at least one insulin antibody in the test strip.

In an embodiment, at least one insulin antibody is directly or indirectly bound to a gold nanoparticle. In the case of indirect binding of an insulin antibody to a gold nanoparticle such binding may include but is not limited to a biotinylated insulin antibody and a gold nanoparticle coated in biotin binding protein, the latter including but not limited to streptavidin.

In an embodiment, the method further comprises determining a quantitative or semi-quantitative concentration of insulin in the biological fluid sample from an equine mammal.

In an embodiment, the method further comprises diagnosing insulin dysregulation (ID), insulin resistance (IR), hyperinsulinemia or Equine Metabolic Syndrome (EMS) in the equine animal.

In an embodiment, the lateral flow assay test device is configured to be read by, at least one of, a visualization chart, a calibrated electronic reader, and an external calibrated electronic reader.

In an embodiment, the method further comprises treating insulin dysregulation (ID), insulin resistance (IR), hyperinsulinemia, Equine Metabolic Syndrome (EMS) or Pituitary Pars Intermedia Dysfunction (PPID) in equine through diet, exercise, nutraceuticals, and pharmaceuticals, or a combination thereof.

In some embodiments, the lateral flow assay strip is configured to be read by, at least one of, a visualization chart, a calibrated electronic reader, and an external calibrated electronic reader. In some embodiments, at least one insulin antibody is conjugated to a gold nanoparticle.

Some embodiments describe a lateral flow assay test device including a body having a sample receiving zone and an opposite zone and comprising a plurality of sandwiched layers including a top layer and a bottom layer whereby allowing a sample fluid to flow from the sample receiving end toward the opposite end through a conjugate pad, the conjugate pad comprising an insulin antibody conjugated to a gold nanoparticle. In some embodiments, the insulin antibody is insulin antibody E2E3. In some embodiments, the lateral flow assay test device further includes a capture antibody. In some embodiments, the capture antibody is antibody 2D11. In some embodiments, the plurality of sandwiched layers comprises a nitrocellulose membrane. In some embodiments, the plurality of sandwiched layers comprises a blood filter pad. In some embodiments, the blood filter pad comprises glass fibers, microglass fibers, cotton fibers, or a combination thereof. In some embodiments, the blood filter pad has a thickness of about 300 μm to about 500 μm. In some embodiments, the lateral flow assay test device further comprises at least one of a conjugate pad, a wick pad, a detection region, a control region, a control agent, and a detection agent.

Some embodiments include a lateral flow assay test device comprising, a flow path configured to receive a whole blood sample premixed with a chase buffer, a sample receiving zone coupled to the flow path, wherein the flow path comprises a blood filter pad directly below the sample receiving zone, a capture zone coupled to the flow path downstream of the sample receiving zone and comprising a capture antibody capable of immobilizing the target analyte, the target analyte previously having been bound by the detection antibody that is conjugated with a gold nanoparticle, a control zone coupled to the capture zone configured to detect gold nanoparticle conjugated insulin detection antibody that has not previously bound to an insulin molecule. In some embodiments, the insulin detection antibody is insulin antibody E2E3. In some embodiments, the insulin capture antibody is antibody 2D11. In some embodiments, the blood filter pad comprises glass fibers, microglass fibers, cotton fibers, or a combination thereof. In some embodiments, the blood filter pad has a thickness of about 300 μm to about 500 μm.

Some embodiments include a method for detecting insulin in a liquid composition. In some embodiments, the method comprises providing a lateral flow assay test device as described herein, contacting the liquid composition with a chase buffer to form a testing sample; and contacting the testing sample with a receiving zone of the lateral test assay test device, allowing the liquid composition to move from the sample receiving zone to the opposite zone, wherein the absence of insulin in the liquid composition is indicated by absence of a test line or band in the capture region of the test strip. In some embodiments, the liquid composition flow rate is about 30 sec/cm to about 40 sec/cm.

Some embodiments include a method for detecting insulin in a whole blood sample. In some embodiments, the method comprises providing a lateral flow assay test device as described herein, contacting the whole blood sample with a chase buffer to form a testing sample; and contacting the testing sample with a receiving zone of the lateral test assay test device, allowing the liquid composition to move from the sample receiving zone to the capture zone, detecting a signal on the capture zone, wherein the presence of insulin is indicated by a signal in the capture zone. In some embodiments, the liquid composition flow rate is about 30 sec/cm to about 40 sec/cm.

Any embodiment is independently combinable, in whole or in part, with any other embodiment or aspect, in whole or in part.

### BRIEF DESCRIPTION OF THE DRAWINGS

FIG. 1A illustrates an embodiment of a top view of a lateral flow assay system housing with sample port and viewing window. FIG. 1B illustrates an embodiment of a cross-section of a lateral flow assay test strip with the multiple layers or components identified.

FIG. 2A illustrates a streptavidin coated gold nanoparticle and a biotinylated detection antibody. FIG. 2B illustrates a biotinylated detection antibody conjugated to a streptavidin coated gold nanoparticle. FIG. 2C illustrates an insulin molecule that has been bound by a biotinylated detection antibody conjugated to a streptavidin coated gold nanoparticle. FIG. 2D illustrates a sandwich that forms in the capture region or at the test line and which includes an insulin molecule that has been bound by both an insulin capture antibody and a biotinylated detection antibody conjugated to a streptavidin coated gold nanoparticle.

FIG. 3A illustrates a comparison table of equine insulin lateral flow assay test line intensities (millivolts) from insulin positive [10 ng/ml (288 uU/mL)] and insulin negative (0 ng/ml) equine plasma samples using different insulin antibody clones as the capture antibody and detector antibody, respectively. FIG. 3B illustrates an image of equine insulin lateral flow assays performed on insulin positive and insulin negative equine plasma samples.

FIG. 4 is a graphical representation of the correlations of insulin lateral flow assays with Cornell radioimmunoassays for 15 equine plasma samples with four different lateral flow assay detection antibody conjugation protocols.

FIG. 5 is a graphical representation of the effect of chase buffer composition on lateral flow assay signal correlation with equine plasma insulin concentration.

FIG. 6A illustrates an image of equine insulin lateral flow assay test strips with different sample or blood filter pads or combinations thereof. FIG. 6B is a graphical comparison of equine insulin lateral flow assays using two different sample or blood filter pads.

FIG. 7 illustrates an image of equine insulin lateral flow assays displaying an increase in test line intensity (from left to right) with increasing concentration of insulin in equine plasma samples.

### DETAILED DESCRIPTION

Lateral flow assay systems, test devices, and methods to improve detection of analytes of interest in a sample are described herein.

### Definitions

Unless defined otherwise, all technical and scientific terms used herein have the same meaning as is commonly understood by one of ordinary skill in the art to which this invention belongs.

As used herein the term “about” can mean within 1 or more standard deviation per the practice in the art. Alternatively, “about” can mean a range of up to 20%, up to 10%, or up to 5%. In certain embodiments, “about” can mean a range of up to 5%. When particular values are provided in the specification and claims the meaning of “about” should be assumed to be within an acceptable error range for that particular value.

As used herein, “analyte” generally refers to a substance to be detected. For instance, analytes may include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles, and metabolites of or antibodies to any of the above substances. Specific examples of some analytes include ferritin; creatinine kinase MB (CK-MB); human chorionic gonadotropin (hCG); digoxin; phenytoin; phenobarbital; carbamazepine; vancomycin; gentamycin; theophylline; valproic acid; quinidine; luteinizing hormone (LH); follicle stimulating hormone (FSH); estradiol, progesterone; C-reactive protein (CRP); lipocalins; IgE antibodies; cytokines; interferon-induced GTP-binding protein (also referred to as myxovirus (influenza virus) resistance 1, MX1, MxA, IFI-78K, IFI78, MX, MX dynamin like GTPase 1); procalcitonin (PCT); glycated hemoglobin (Gly Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryonic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Additional analytes may be included for purposes of biological or environmental substances of interest.

As used herein, the term “sample” includes, but is not limited to, a fluid, which may comprise insulin, a solution, which may comprise insulin, and a biological sample obtained from a human or animal subject. Biological samples include but are not limited to saliva, serum, blood, urine, or exhaled breath condensate. In certain embodiments, the sample may be fresh. It will be appreciated that a fresh sample includes, but is not limited to, a sample obtained from a subject and that is subjected to insulin detection by methods herein described within several seconds, for example, less than about 1 to about 3 minutes, after the sample is obtained. In related embodiments, a sample is directly applied to a sample region, wherein the sample is not pre-treated and/or purified prior to application to the sample region. In certain embodiments, the sample may be a stored sample. It will be appreciated that a stored sample may have been prepared and/or obtained from a subject and subjected to storage, for example in a refrigerator or freezer prior to subjecting the sample to insulin detection by methods herein described. In some embodiments, the sample may be phosphate buffered saline (PBS) spiked with different concentrations of insulin. In certain embodiments, a sample may be applied to a sample region wherein the sample is not subjected to any processing (for example, dilution, filtration, concentration) prior to application to the sample region. In certain embodiments, a sample may be concentrated prior to application to a sample region. In certain embodiments, a sample may be diluted or mixed with a chemical solution, including but not limited to, a chase or running buffer, prior to application to a sample region. In certain embodiments, a sample may be filtered prior to application to a sample region. In certain embodiments wherein the sample is blood or a mixture of blood with chase or running buffer, a lateral flow assay device may further comprise a sample or blood filter membrane in or applied to the sample region.

The term “specific binding partner (or binding partner)” refers to a member of a pair of molecules that interacts by means of specific, noncovalent interactions that depend on the three-dimensional structures of the molecules involved. Typical pairs of specific binding partners include antigen/antibody, hapten/antibody, hormone/receptor, nucleic acid strand/complementary nucleic acid strand, substrate/enzyme, inhibitor/enzyme, carbohydrate/lectin, biotin/(strept) avidin, receptor/ligands, and virus/cellular receptor, or various combinations thereof.

As used herein, the terms “immunoglobulin” or “antibody” refer to proteins that bind a specific antigen. Immunoglobulins or antibodies include, but are not limited to, polyclonal, monoclonal, chimeric, and humanized antibodies, Fab fragments, F(ab′) 2 fragments, and includes immunoglobulins of the following classes: IgG, IgA, IgM, IgD, IbE, and secreted immunoglobulins (slg). Immunoglobulins generally comprise two identical heavy chains and two light chains. However, the terms “antibody” and “immunoglobulin” also encompass single chain antibodies and two chain antibodies. For simplicity, through the specification the terms “labeled antibody” or “capture antibody” is used, but the term antibody as used herein refers to the antibody as a whole or any fragment thereof. Thus, it is contemplated that when referring to a labeled antibody that specifically binds analyte of interest, the term refers to a labeled antibody or fragment thereof that specifically binds an analyte of interest. Similarly, when referring to a capture antibody, the term refers to a capture antibody or fragment thereof that specifically binds to the analyte of interest.

As used herein, an “ancillary binding partner” is a specific binding partner that binds to the specific binding partner of an analyte. For example, an ancillary specific binding partner may include an antibody specific for another antibody, for example, goat anti-human antibody. Lateral flow devices described herein can include a “detection area” or “detection zone” that is an area that includes one or more capture area or capture zone and that is a region where a detectable signal may be detected. Lateral flow devices described herein can include a “capture area” that is a region of the lateral flow device where the capture reagent is immobilized. Lateral flow devices described herein may include more than one capture area. In some cases, a different capture reagent will be immobilized in different capture areas (such as a first capture reagent at a first capture area and a second capture agent at a second capture area). Multiple capture areas may have any orientation with respect to each other on the lateral flow substrate; for example, a first capture area may be distal or proximal to a second (or other) capture area along the path of fluid flow and vice versa. Alternatively, a first capture area and a second (or other) capture area may be aligned along an axis perpendicular to the path of fluid flow such that fluid contacts the capture areas at the same time or about the same time.

As used herein, “Equine Metabolic Syndrome” is the presence of insulin dysregulation, insulin resistance, obesity and/or regional adiposity. The Equine Metabolic Syndrome phenotype may also comprise dyslipidemia, dysadipokinemia and/or hypertension. The syndrome can be described as a combination of medical disorders that increase the risk of developing associated pathologies, e.g., laminitis. Equine Metabolic Syndrome might also be associated with other disorders like hepatic lipidosis or infertility.

As used herein, “Pituitary Pars Intermedia Dysfunction” is a common disease of older horses and ponies. Hypothalamic dopaminergic neurodegeneration results in an elevated adrenocorticotropic hormone (ACTH) production in the Pituitary Pars Intermedia and leads to hyperadrenocorticism. Clinical signs include hirsutism (a long, often curly coat that may not shed), polydipsia/polyuria, excessive sweating, weight loss, muscle wasting, regional fat deposits, lethargy, infections e.g., sinusitis and/or laminitis.

As used herein, “Equine animal” may be used interchangeably with the term “equine” and encompasses any member of the genus Equus. It encompasses any horse or pony, the taxonomic designations Equus ferns and/or Equus caballus, and/or the subspecies Equus ferns caballus.

### Lateral Flow Assay System

In some aspects, a lateral flow assay test system may include a lateral flow assay test device, a system housing, a reader, a data analyzer, and combinations thereof.

In some embodiments, a lateral flow assay test device may include a sample port (also referred to as a sample receiving zone) where a fluid sample is introduced to a test strip. In another embodiment, the sample may be introduced to the sample port by external application, as with a dropper or other applicator. The sample may be poured or expressed onto the sample port. In another example, the sample port may be directly immersed in the sample, such as when a test strip is dipped into a container holding a sample. In some embodiments, the sample port comprises an insulin probe. In some embodiments, the insulin probe is an aptamer specific for insulin. In some embodiments, the test strip comprises at least one of a sample pad, a blood filter, a conjugate pad, a nitrocellulose membrane, a wick pad, a detection region, a control region, a control agent, an insulin antibody, a nanoparticle, and a detection agent.

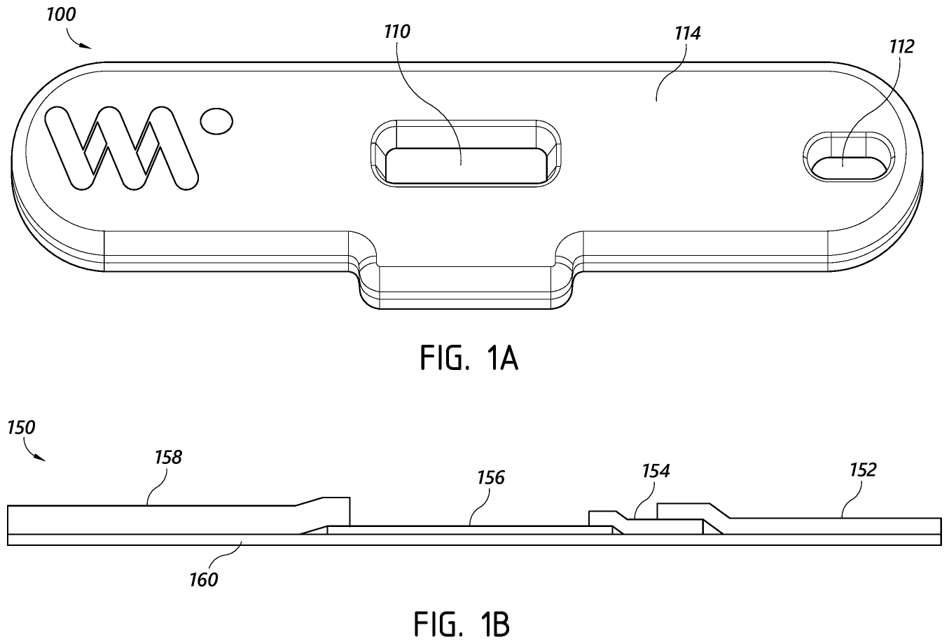

Referring to FIG. 1A, FIG. 1A illustrates an embodiment of the lateral flow assay system housing 100 that contains the test strip 150 with the locations of the sample port 112 and viewing window 110 in the top portion of the housing 114. FIG. 1B is a detailed cross-section of the test strip 150 illustrating the configuration of the individual components or layers that comprise the flow path of the test strip 150. In an embodiment, as shown in FIG. 1A, the sample port 112 is an opening in the system housing 114 where a sample is applied to the lateral flow assay, and the viewing window 110 is a second opening in the system housing 114 where control and test line development and reading occur. In some embodiments, as shown in FIG. 1B, there is a blood filter pad or sample pad 152 situated at a first end of a test strip 150 (right end as illustrated). The blood filter pad or sample pad 152 sits on top of a conjugate pad 154 which contains at least one conjugate that specifically binds the analyte of interest. The conjugate pad 154 sits on top of the nitrocellulose membrane 156 which contains a capture region and control region. A wick pad 158 sits on top of the nitrocellulose membrane 156 on the opposite end (left end as illustrated) of the nitrocellulose membrane 156. A backing card 160 supports the layered components of the test strip that remain in fluid contact with one another. In some embodiments, there are two blood filter pads or sample pads 152 or a combination thereof placed on top of each other. In some embodiments, the conjugate pad 154 includes an insulin detection antibody conjugated with a gold nanoparticle. In some embodiments, a biotinylated insulin detection antibody is conjugated to a gold nanoparticle that is coated in streptavidin. In some embodiments the nitrocellulose membrane 154 includes an insulin capture antibody that immobilizes the analyte of interest and its gold nanoparticle label.

Lateral flow assay test devices described herein can include a solid support or substrate. Suitable solid supports include but are not limited to nitrocellulose, the walls of wells of a reaction tray, multi-well plates, test tubes, polystyrene beads, magnetic beads, membranes, and microparticles (such as latex particles). Any suitable porous material with sufficient porosity to allow access by labeled conjugates and a suitable surface affinity to immobilize capture agents can be used in lateral flow devices described herein. For example, the porous structure of nitrocellulose has excellent absorption and adsorption qualities for a wide variety of reagents, for instance, capture agents. Nylon possesses similar characteristics and is also suitable. Microporous structures are useful, as are materials with gel structure in the hydrated state.

The surface of a solid support may be activated by chemical processes that cause covalent linkage of an agent (e.g., a capture reagent) to the support. As described herein, the solid support can include a conjugate pad. Many other suitable methods may be used for immobilizing an agent (e.g., a capture reagent) to a solid support including, without limitation, ionic interactions, hydrophobic interactions, covalent interactions and the like. Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

Further examples of useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

In some embodiments, lateral flow assay test device may include porous solid supports, such as nitrocellulose, in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In some embodiments, the flow rate is about 35 sec/cm (i.e., 140 sec/4 cm) to about 37.5 sec/cm (i.e., 150 sec/4 cm). In specific embodiments of devices described herein, the flow rate is about 35 sec/cm (i.e., 140 sec/4 cm). In other embodiments of devices described herein, the flow rate is about 37.5 sec/cm (i.e., 150 sec/4 cm).

In some embodiments, the lateral flow device may include a label. Labels can take many different forms, including a molecule or composition bound or capable of being bound to an analyte, analyte analog, detector reagent, ancillary binding partner or a specific binding partner that is detectable by spectroscopic, photochemical, biochemical, immunochemical, electrical, optical or chemical means. Examples of labels include enzymes, colloidal gold particles (also referred to as gold nanoparticles), colored latex particles, radioactive isotopes, co-factors, ligands, chemiluminescent or fluorescent agents, protein-adsorbed silver particles, protein-adsorbed iron particles, protein-adsorbed copper particles, protein-adsorbed selenium particles, protein-adsorbed sulfur particles, protein-adsorbed tellurium particles, protein-adsorbed carbon particles, and protein-coupled dye sacs. The attachment of a compound (e.g., a detector reagent) to a label can be through covalent bonds, adsorption processes, hydrophobic and/or electrostatic bonds, as in chelates and the like, or combinations of these bonds and interactions and/or may involve a linking group. The lateral flow assays and devices described herein include separation membranes for removing confounding components, including components that have the same or similar optical characteristics as the optical characteristics of the label. For example, red blood cells, having hemoglobin present, have a similar optical characteristic as gold nanoparticles. Thus, in some embodiments, when gold nanoparticles are used for detecting a signal, red blood cells can be separated using the separation membrane according to the present disclosure. Similarly, other metal nanoparticles, including silver, platinum, copper, palladium, ruthenium, rhenium, or other metal nanoparticles generate specific signals whose detection may be similarly enhanced by removing confounding components from a sample in accordance with the present disclosure.

In some embodiments, the insulin antibody is an insulin peptide antibody. In some embodiments, the insulin antibody is an insulin growth hormone antibody. In some embodiments, the insulin antibody is insulin antibody 2D11. Insulin antibody 2D11, also referred to as 2D11-H5, is a high quality monoclonal insulin antibody. Insulin antibody 2D11 is commercially available as both the non-conjugated anti-insulin antibody form, as well as multiple conjugated forms of anti-insulin antibody, including agarose, HRP, PE, FITC and multiple Alexa Fluor® conjugates. In some embodiments, the insulin antibody is E2E3. Insulin antibody E2E3 is also referred to as INS04. Insulin antibody E2E3 is a mouse monoclonal antibody. In some embodiments, the insulin antibody is not an anti-FAM monoclonal antibody.

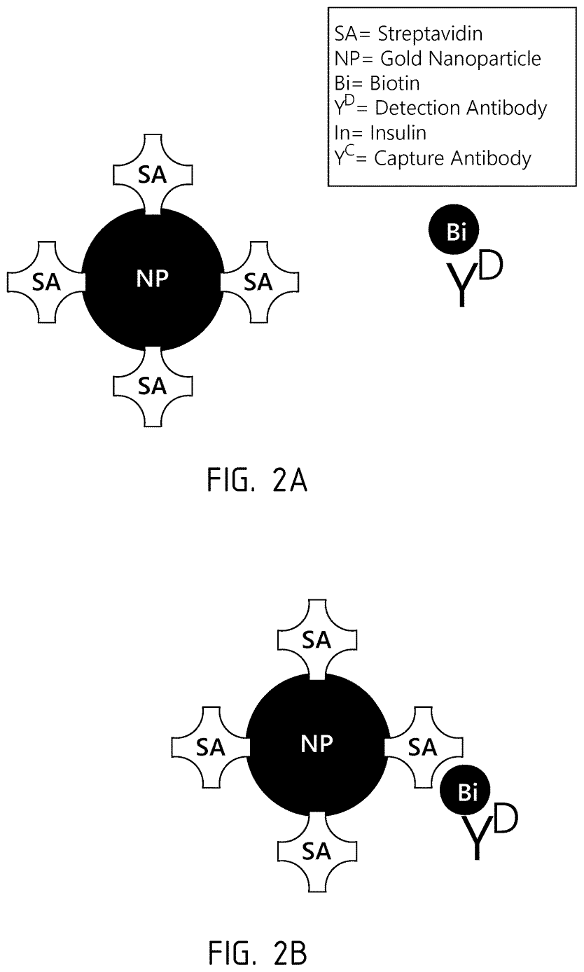

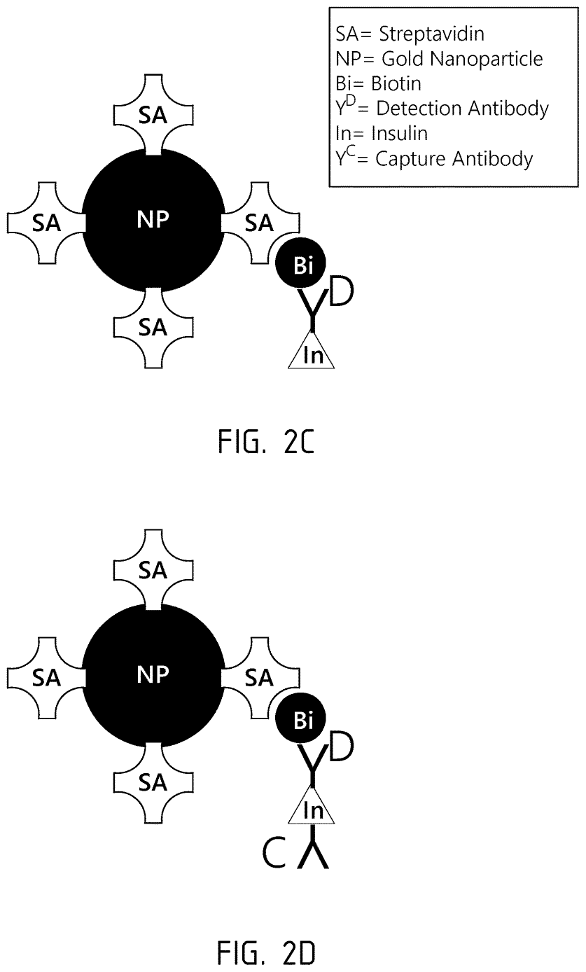

In other embodiments, the conjugate pad contains the insulin detection antibody and the nitrocellulose membrane contains the insulin capture antibody. In some embodiments, now referring to FIG. 2A, a gold nanoparticle (NP) is coated in streptavidin (SA) and the detection antibody (Y^{D}) is biotinylated. In some embodiments, referring to FIG. 2B, a biotinylated (Bi) detection antibody (Y^{D}) is conjugated to a streptavidin (SA) coated gold nanoparticle (NP). In some embodiments, referring to FIG. 2C, an insulin molecule (In) is bound or detected by a biotinylated (Bi) detection antibody (Y^{D}) that has previously been conjugated to a streptavidin (SA) coated gold nanoparticle (NP). In some embodiments, referring to FIG. 2D, a sandwich forms in the capture region or at the test line and includes an insulin molecule (In) that has been bound by both an insulin capture antibody (Y^{c}) and a biotinylated (Bi) insulin detection antibody (Y^{D}) conjugated to a streptavidin (SA) coated gold nanoparticle (NP). In some embodiments, an insulin antigen in a fluid sample or liquid composition is bound by a gold nanoparticle labeled detection antibody in the conjugate pad and is further bound by a capture antibody in the nitrocellulose membrane to form a sandwich, the sandwich including the gold nanoparticle labeled insulin detection antibody, the insulin antigen, and the insulin capture antibody, with this sandwich being immobilized in the capture region of the lateral flow assay, the latter being apparent in the viewing window of the system housing as a test line. In some embodiments, the sandwich is formed after the addition of a chase buffer or a running buffer. In some embodiments, the analyte or antigen includes insulin. In some embodiments, the first binding partner includes an insulin detection antibody. In some embodiments, the insulin detection antibody is E2E3. In some embodiments, the insulin detection antibody is conjugated to a reporter or tag. In some embodiments, the reporter or tag is a nanoparticle. In some embodiments, the nanoparticle is a gold nanoparticle. In some embodiments, the insulin detection antibody is biotinylated. In some embodiments, the gold nanoparticle is coated in streptavidin. In some embodiments, the biotinylated detection antibody is indirectly conjugated to streptavidin coated gold nanoparticles through the binding of biotin and streptavidin. In some embodiments, the detection antibody is directly bound to a nanoparticle reporter or tag. In some embodiments, the capture antibody is an insulin antibody. In some embodiments, the capture antibody is insulin antibody 2D11. In some embodiments, the chase buffer or running buffer is added to permit flow of insulin bound by the detection antibody with gold nanoparticle reporter attached to the capture region where a test line is developed. In some embodiments, the chase buffer or running buffer is added to whole blood before being adding to the sample port. In some embodiments, whole blood is premixed with chase buffer or running buffer to form a testing sample that is added to the sample port.

In some embodiments, the blood filter is configured for whole blood filtering. In some embodiments, the blood filter is a blood filter pad. In some embodiments, the blood filter pad is a nitrocellulose membrane. In some embodiments, the blood filter pad separates plasma from whole blood samples in lateral flow applications while retaining bloods cells and allowing serum to flow rapidly. In some embodiments, the blood filter pad comprises glass fibers, microglass fibers, cotton fibers, or a combination thereof. In some embodiments, the blood filter pad has a thickness of about 300 μm to about 500 μm. In some embodiments, the blood filter pad is a Cyctosep® HV plus 1668. In some embodiments, the blood filter pad is type FR-1 blood filter pad.

In some embodiments, the lateral flow device may include capture agents that are immobilized such that movement of the capture agent is restricted during normal operation of the lateral flow device. For example, movement of an immobilized capture agent is restricted before and after a fluid sample is applied to the lateral flow device. Immobilization of capture agents can be accomplished by physical means such as barriers, electrostatic interactions, hydrogen bonding, bioaffinity, interactions, or combinations thereof.

In some embodiments, the labeled conjugate (or more than one labeled conjugate, if such is the case) may be integrated on the conjugate pad by physical or chemical bonds. The sample solubilizes the labeled conjugate after the sample is added to the sample reservoir, releasing the bonds holding the labeled conjugate to the conjugate pad. The labeled conjugate binds to the analyte of interest, if present in the sample, forming a complex.

In some embodiments, the separation membrane may separate components of a sample based on size and/or affinity of components to the membrane, while allowing objects of interest to pass through the membrane and flow in the fluid path to a detection zone of the assay. In one example, a separation membrane of the present disclosure allows passage of smaller components of a sample but does not allow passage of larger components (such as confounding components) of a sample. The characteristics of the separation membrane can be optimized to prevent passage of the larger confounding components typically expected to be present in a fluid sample. In another example, a separation membrane of the present disclosure includes affinity agents that bind (specifically or non-specifically) to components (such as confounding components) of a sample, but does not bind to objects of interest (such as analytes of interest) in the sample. In a further example, a separation membrane of the present disclosure retains undesirable components in a sample based on both size and affinity characteristics of the components.

Embodiments of the present disclosure can include a separation membrane specifically selected and designed to retain components that interfere with detection of a particular analyte of interest present at a concentration near the detection threshold of a conventional measurement system (where signals may fall at or below the detection threshold and yield a false negative test result). Thus, embodiments of the present disclosure can increase accuracy of a lateral flow device by improving detection of signals at the detection zone that would ordinarily fall below the detection threshold of a conventional measurement system.

Embodiments of the present disclosure can include a separation membrane specifically selected and designed to retain components that interfere with detection of a particular labeled conjugate. One example type of interference occurs when a confounding component has an optical characteristic that is substantially the same or similar to an optical characteristic of the labeled conjugate in the sandwich structure formed in the detection zone. In one embodiment, the labeled conjugate includes a gold nanoparticle, which generates a signal with optical properties similar to optical properties of red blood cells in a blood sample. For example, the gold nanoparticle may generate a signal at the same or similar wavelength of light as a red blood cell. Embodiments of the present disclosure reduce or eliminate interference from confounding components, such as but not limited to red blood cells in a sample, by retaining or capturing the confounding components at a separation membrane, such that the optical characteristics of the red blood cells do not interfere with detection of signals generated at the detection zone.

In some embodiments, the system housing may be made of any one of a wide variety of materials, including plastic, metal, or composite materials. The system housing forms a protective enclosure for components of the diagnostic test system. The system housing also defines a receptacle that mechanically registers the test strip with respect to the reader. The receptacle may be designed to receive any one of a wide variety of different types of test strips. In some embodiments, the system housing is a portable device that allows for the ability to perform a lateral flow assay in a variety of environments, including on the bench, in the field, in the home, or in a facility for domestic, commercial, or environmental applications.

The system housing of any of the lateral flow assay test systems described herein, including the top housing or the base housing, may be made with any suitable material, including, for example, vinyl, nylon, polyvinyl chloride, polypropylene, polystyrene, polyethylene, polycarbonates, polysulfanes, polyesters, urethanes, or epoxies. The housing may be prepared by any suitable method, including, for example, by injection molding, compression molding, transfer molding, blow molding, extrusion molding, foam molding, thermoform molding, casting, layer deposition, or printing.

In some embodiments, a reader may include one or more optoelectronic components. The one or more optoelectronic components may be for optically inspecting the exposed areas of the detection zone of the test strip, and capable of detecting multiple capture zones within the detection zone. In some embodiments, the reader includes at least one light source and at least one light detector. In some embodiments, the light source may include a semiconductor light-emitting diode and the light detector may include a semiconductor photodiode. Depending on the nature of the label that is used by the test strip, the light source may be designed to emit light within a particular wavelength range or light with a particular polarization. For example, if the label is a fluorescent label, such as a quantum dot, the light source would be designed to illuminate the exposed areas of the capture zone of the test strip with light in a wavelength range that induces fluorescent emission from the label. Similarly, the light detector may be designed to selectively capture light from the exposed areas of the capture zone. For example, if the label is a fluorescent label, the light detector would be designed to selectively capture light within the wavelength range of the fluorescent light emitted by the label or with light of a particular polarization. On the other hand, if the label is a reflective-type label, the light detector would be designed to selectively capture light within the wavelength range of the light emitted by the light source. To these ends, the light detector may include one or more optical filters that define the wavelength ranges or polarizations axes of the captured light. A signal from a label can be analyzed, using visual observation or a spectrophotometer to detect color from a chromogenic substrate; a radiation counter to detect radiation, such as a gamma counter for detection of ¹²⁵I; or a fluorometer to detect fluorescence in the presence of light of a certain wavelength. Where an enzyme-linked assay is used, quantitative analysis of the amount of an analyte of interest can be performed using a spectrophotometer. Lateral flow assays described herein can be automated or performed robotically, if desired, and the signal from multiple samples can be detected simultaneously. Furthermore, multiple signals can be detected for plurality of analytes of interest, including when the label for each analyte of interest is the same or different. In some embodiments, the reader may include a camera-based reader.

In some embodiments, signals generated by assays may be in the context of an optical signal generated by reflectance-type labels (such as but not limited to gold nanoparticle labels and different-colored latex particles). Although embodiments of the present disclosure are described herein by reference to an “optical” signal, it will be understood that assays described herein can use any appropriate material for a label in order to generate a signal, including but not limited to fluorescence-type latex bead labels that generate fluorescence signals and magnetic nanoparticle labels that generate signals indicating a change in magnetic fields associated with the assay.

In some embodiments, the data analyzer processes the signal measurements that are obtained by the reader. In general, the data analyzer may be implemented in any computing or processing environment, including in digital electronic circuitry or in computer hardware, firmware, or software. In some embodiments, the data analyzer includes a processor (e.g., a microcontroller, a microprocessor, or ASIC) and an analog-to-digital converter. The data analyzer can be incorporated within the housing of the diagnostic test system. In other embodiments, the data analyzer is located in a separate device, such as a computer, that may communicate with the diagnostic test system over a wired or wireless connection. The data analyzer may also include circuits for transfer of results via a wireless connection to an external source for data analysis or for reviewing the results.

In general, the results indicator may include any one of a wide variety of different mechanisms for indicating one or more results of an assay test. In some implementations, the results indicator includes one or more lights (e.g., light-emitting diodes) that are activated to indicate, for example, the completion of the assay test. In other implementations, the results indicator includes an alphanumeric display (e.g., a two or three character light-emitting diode array) for presenting assay test results.

Test systems described herein can include a power supply that supplies power to the active components of the diagnostic test system, including the reader, the data analyzer, and the results indicator. The power supply may be implemented by, for example, a replaceable battery or a rechargeable battery. In other embodiments, the diagnostic test system may be powered by an external host device (e.g., a computer connected by a USB cable).

### Lateral Flow Assay

In some aspects, a method of testing disease in a subject may comprise obtaining a fluid sample from an animal and contacting the fluid sample with a lateral flow assay test system.

In some embodiments, a subject may be an animal or a human. The animal can be a farm animal such as a pig, cow, horse, sheep or goat. The animal can be a companion animal such as a dog or cat. The animal can be a laboratory animal such as a rabbit, mouse or rat. In other embodiments, the animal is an equine mammal.

In some embodiments, the fluid sample may be any suitable sample liquid. In some embodiments, the liquid sample can be body fluid sample, such as a whole blood, a serum, a plasma, a urine sample or an oral fluid. Such body fluid sample can be used directly or can be processed, e.g., enriched, purified, or diluted, before use. In other embodiments, the liquid sample can be a liquid extract, suspension or solution derived from a solid or semi-solid biological material such as a phage, a virus, a bacterial cell, a eukaryotic cell, a fungal cell, a mammalian cell, a cultured cell, a cellular or subcellular structure, cell aggregates, tissue or organs. In specific embodiments, the sample liquid is obtained or derived from a mammalian or human source. In still other embodiments, the liquid sample is a sample derived from a biological, a forensics, a food, a biowarfare, or an environmental source. In other embodiments, the sample liquid is a clinical sample, e.g., a human or animal clinical sample. In still other embodiments, the sample liquid is a man-made sample, e.g., a standard sample for quality control or calibration purposes.

In some embodiments, the method can be used to detect the presence, absence and/or amount of an analyte in any suitable sample liquid. In some embodiments, the present test devices are used to detect the presence or absence of an analyte in any suitable sample liquid, i.e., to provide a yes or no answer. In other embodiments, the present test devices are used to quantify or semi-quantify the amount of an analyte in a liquid sample. In some embodiments, a lateral flow device system can detect, identify, and in some cases quantify a biologic. A biologic includes chemical or biochemical compounds produced by a living organism, including a prokaryotic cell line, a eukaryotic cell line, a mammalian cell line, a microbial cell line, an insect cell line, a plant cell line, a mixed cell line, a naturally occurring cell line, or a synthetically engineered cell line. A biologic can include large macromolecules such as proteins, polysaccharides, lipids, and nucleic acids, as well as small molecules such as primary metabolites, secondary metabolites, and natural products.

In some embodiments, the lateral flow assay system described herein are highly sensitive to an analyte of interest present in a sample, including to one or more analyte of interest present at significantly different concentrations, such as at high concentrations (in the 10s to 100s of μg/mL) and at low concentrations (in the 1s to 10s of pg/mL). “Sensitivity” refers to the proportion of actual positives that are correctly identified as such (for example, the percentage of infected, latent, or symptomatic subjects who are correctly identified as having a condition). Sensitivity may be calculated as the number of true positives divided by the sum of the number of true positives and the number of false negatives.

For example, aspects of the lateral flow assays described herein include contacting the lateral flow assay system with a volume of raw, unprocessed sample of between 10 μL and 1000 μL. In an embodiment, the raw, unprocessed sample is a whole blood sample.

In some embodiments, the lateral flow assay system can measure the presence and concentration of multiple analytes of interest. In some embodiments, the lateral flow assay system can determine significantly different concentrations in a single, undiluted, unprocessed sample. In some embodiments, the lateral flow assay system can measure a single test event. In some embodiments, the single lateral flow assay can measure one or more analytes. In some embodiments, the lateral flow assay system can measure the presence and concentration of multiple analytes of interest present in a sample at different concentrations without ever diluting the sample.

Depending on the type of specimen and the source from which the specimen is taken, a specimen may be processed, treated, or prepared to obtain a sample in a format that is suitable to be applied to a lateral flow assay system. The source of the specimen can be a biological source, an environmental source, or any other source suspected of including an analyte of interest. Embodiments of the present disclosure can detect analytes of interest in a specimen that has not been processed prior to contacting the lateral flow device with the specimen. In one non-limiting example, a specimen that has not been processed, treated, or prepared is applied to a lateral flow device according to the present disclosure. In this example, the raw specimen obtained from the original source is not processed into a sample before applying the raw specimen to the lateral flow device of the present disclosure. Although reference is made throughout the present disclosure to a “sample” being applied to a lateral flow device, it will be understood that such sample can include a raw specimen that has not been processed or prepared into a conventional sample format.

In one non-limiting example, the sample is a raw sample that includes all components as directly obtained from a source, including but not limited to a biological subject. In one embodiment, the raw sample is any unmodified collected blood sample, referred to herein as a whole blood sample. In this non-limiting example, a separation membrane according to the present disclosure includes a plasma separation membrane, capable of separating components of the whole blood sample based on the size of the component. The whole blood sample contacts the plasma separation membrane. Confounding components in the whole blood sample, such as red blood cells, are retained on or captured in the plasma separation membrane, because the red blood cells are too large to pass through the plasma separation membrane. Plasma, which may include analyte of interest, passes through the plasma separation membrane, and flows onto the assay test strip of the present disclosure.

In some embodiments, the analyte of interest, if present, contacts labeled conjugate, which includes a label and an antibody or fragment thereof that specifically binds the analyte of interest. The labeled conjugate, now bound to analyte of interest, flows through the assay test strip to a detection zone, wherein immobilized capture agent binds analyte of interest. If present, analyte of interest, bound to labeled conjugate, is captured by the immobilized capture agent in the detection zone to form a “sandwich” structure. The sandwich structure may generate a signal above a detection threshold of a measurement system, indicating the presence and in some cases the quantity of analyte of interest present in the sample. If the analyte of interest is not present in the sample, sandwich structures do not form and a signal is not generated in the detection zone, indicating absence of the analyte of interest.

Some embodiments provided herein relate to methods of using lateral flow assays to detect an analyte of interest in a raw sample. In some embodiments, the method includes providing a lateral flow assay as described herein. In some embodiments, the method includes applying a fluid sample to a lateral flow device described herein.

In some embodiments, applying a sample on the lateral flow device includes applying the sample at the sample port of the lateral flow device. In some embodiments, applying the sample at the sample port includes contacting a sample with a lateral flow assay. A sample may contact a lateral flow assay by introducing a sample to a sample port by external application, as with a dropper or other applicator. In some embodiments, a sample port may be directly immersed in the sample, such as when a test strip is dipped into a container holding a sample. In some embodiments, a sample may be poured, dripped, sprayed, placed, or otherwise contacted with the sample reservoir.

In some embodiments, the method includes separating particulates from the fluid sample by passing the fluid sample through the separation membrane of the sample well, wherein the analyte of interest passes through the separation membrane to the assay strip. In some embodiments, the particulates include confounding components, including for example, red blood cells, particulates, cellular components, or cellular debris, or other components that impede the flow of sample through a device or interfere with a detection signal of a device. The separation membrane may separate components of the sample based on size, affinity to the membrane, or other characteristics as desired.

In some embodiments, the method includes labeling an analyte of interest with a labeled conjugate. The labeled conjugate may include an antibody that specifically binds an analyte of interest and a label. The labeled conjugate can be deposited on a conjugate pad (or label zone) below or downstream of the sample port. The labeled conjugate can be used to determine the presence and/or quantity of analyte that may be present in the sample. Additional labeled conjugates may also be included on the device, where the operator is interested in determining the presence and/or quantity of more analytes of interest. Thus, the device may include a second labeled conjugate that includes a second antibody that specifically binds a second analyte of interest and a label, and the device may also include a third labeled conjugate that includes a third antibody that specifically binds a third analyte of interest and a label, or more, depending on the number of analytes to be analyzed.

In some embodiments, the method includes binding labeled analyte of interest to immobilized capture agents at a detection zone. In some embodiments, the method includes detecting a signal from the labeled analyte of interest bound to the immobilized capture agents in the detection zone. In some embodiments, a buffer is added. In some embodiments, upon addition of a buffer (such as a chase buffer, including HEPES, PBS, TRIS, or any other suitable buffer) the sample, including bound analyte of interest, flows along the fluid front through the lateral flow assay to a detection zone. The detection zone may include a capture zone for capturing each complex (where more than one analyte of interest is to be detected and/or quantified). For example, the detection zone may include a first capture zone for capturing a first complex, a second capture zone for capturing a second complex, and a third capture zone for capturing a third complex. When first complex binds to first capture agent at the first capture zone, a first signal from the label is detected. The first signal may include an optical signal as described herein. The first signal may be compared to values on a dose response curve for the first analyte of interest, and the concentration of first analyte in the sample is determined.

In some embodiments, a sample is obtained from a source, including an environmental or biological source. In some embodiments, the sample is suspected of having one or more analytes of interest. In some embodiments, the sample is not suspected of having any analytes of interest. In some embodiments, a sample is obtained and analyzed for verification of the absence or presence of a plurality of analytes. In some embodiments, a sample is obtained and analyzed for the quantity of a plurality of analyte in the sample. In some embodiments, the quantity of any one of the one or more analytes present in a sample is less than a normal value present in healthy subjects, at or around a normal value present in healthy subjects, or above a normal value present in healthy subjects. In some embodiments, the fluid sample is an undiluted, whole blood sample; an undiluted venous blood sample; an undiluted capillary blood sample; an undiluted, serum sample; or an undiluted plasma sample. In some embodiments, the fluid sample is applied in an amount of 10 to 100 μL.

In some embodiments, the detected signal is an optical signal, a fluorescent signal, or a magnetic signal. In some embodiments, the device further comprises a buffer port. In some embodiments, the method further includes flowing the buffer through the assay strip to the analyte of interest.

In some embodiments, the analyte of interest is present in elevated concentrations. Elevated concentrations of analyte can refer to a concentration of analyte that is above healthy levels. Thus, elevated concentration of analyte can include a concentration of analyte that is 5%, 10%, 15%, 20%, 25%, 30%, 35%, 40%, 50%, 60%, 70%, 80%, 90%, 100%, 125%, 150%, 200%, or greater than a healthy level. In some embodiments, the analyte of interest includes an analyte as described herein. Additional analytes may be included for purposes of biological or environmental substances of interest.

In some embodiments, the subject is diagnosed with a metabolic disorder. The metabolic disorder may be insulin resistance, hyperinsulinemia, and/or a clinical condition or sign associated with insulin resistance and/or hyperinsulinemia. The metabolic disorder or clinical condition or sign of the disorder may be one or more disorders selected from insulin resistance, hyperinsulinemia, impaired glucose tolerance, dyslipidemia, dysadipokinemia, subclinical inflammation, systemic inflammation, low grade systemic inflammation, which also comprises adipose tissue, obesity, regional adiposity, laminitis, vascular dysfunction, hypertension, hepatic lipidosis, atherosclerosis, hyperadrenocorticism, Pituitary Pars Intermedia Dysfunction and/or Equine Metabolic Syndrome. In some embodiments, Equine Metabolic Syndrome may be associated with obesity and/or regional adiposity.

In some aspects, a lateral flow assay test system comprises a volumetric pipette, a chemical reagent solution, wherein the chemical reagent solution is a chase buffer or a running buffer, a lateral flow assay test device, a system housing comprising one or more ports, configured to receive a biological sample, the chase buffer, the running buffer or a combination thereof, and a reader comprising a light source and a light detector, and a data analyzer. In some embodiments, the lateral flow assay test device comprises an insulin antibody. In some embodiments, the insulin antibody is insulin antibody 2D11. In some embodiments, the insulin antibody is E2E3. In some embodiments, the insulin antibody is not an aptamer. In some embodiments, the lateral flow assay test system does not comprise an insulin probe wherein the insulin probe is a polynucleotide. In some embodiments, the insulin probe is not an anti-FAM monoclonal antibody. In some embodiments, the lateral flow assay test device does not comprise a conditioning pad. In some embodiments, the lateral flow assay test device does not comprise a plurality of laminated layers. In some embodiments, the lateral flow assay test device does not comprise a plurality of window frame layers. In some embodiments, the lateral flow assay test device does not comprise oversized particles. In some embodiments, the lateral flow assay test device comprises a gold nanoparticle. In some embodiments, the gold nanoparticle is not covalently bound to a test strip. In some embodiments, the lateral flow assay test device does not comprise a decomplexation region for dissociating analyte-antibody complexes. In some embodiments, the lateral flow assay test device does not comprise one or more immunoreagents to form one or more capturable and detectable immunocomplex(es). In some embodiments, the lateral flow assay test device does not comprise a fluorescent tag or a fluorescent label. In some embodiments, the lateral flow assay test device does not comprise an immunochromatographic label. In some embodiments, the lateral flow assay test device does not comprise one or more CRISPR effector system. In some embodiments, the lateral flow assay test device does not comprise a logic circuit. In some embodiments, the lateral flow assay test device is not a competitive assay-based lateral flow device. In some embodiments, the lateral flow assay test device is not a rolling circle amplification-based lateral flow device. In some embodiments, the lateral flow assay test device is not a liposome signal amplification-based lateral flow device.

### EXAMPLES

The following examples are intended to illustrate details of the disclosure, without thereby limiting it in any manner.

### Example 1. Antibody Pairing Studies in a Direct (Sandwich) Lateral Flow Assay for Equine Insulin

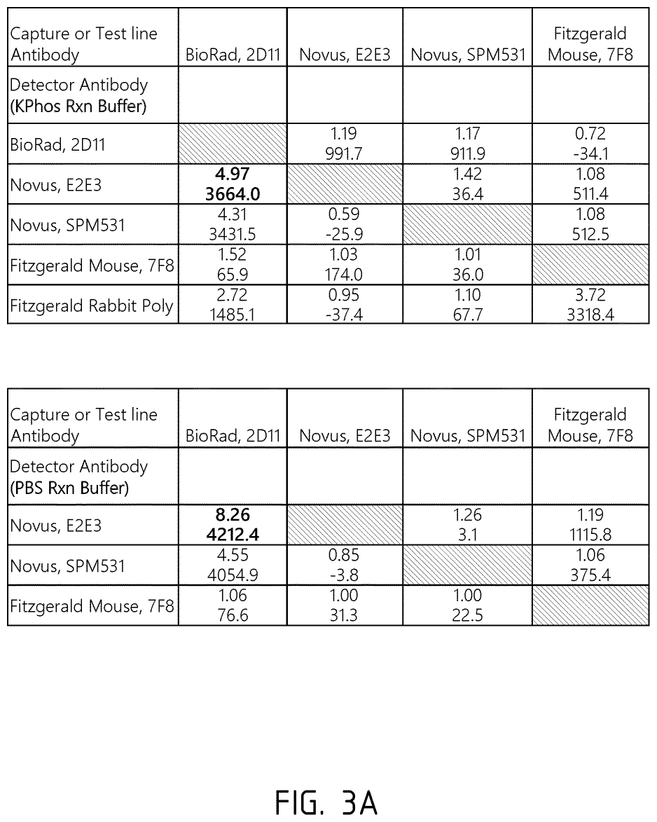

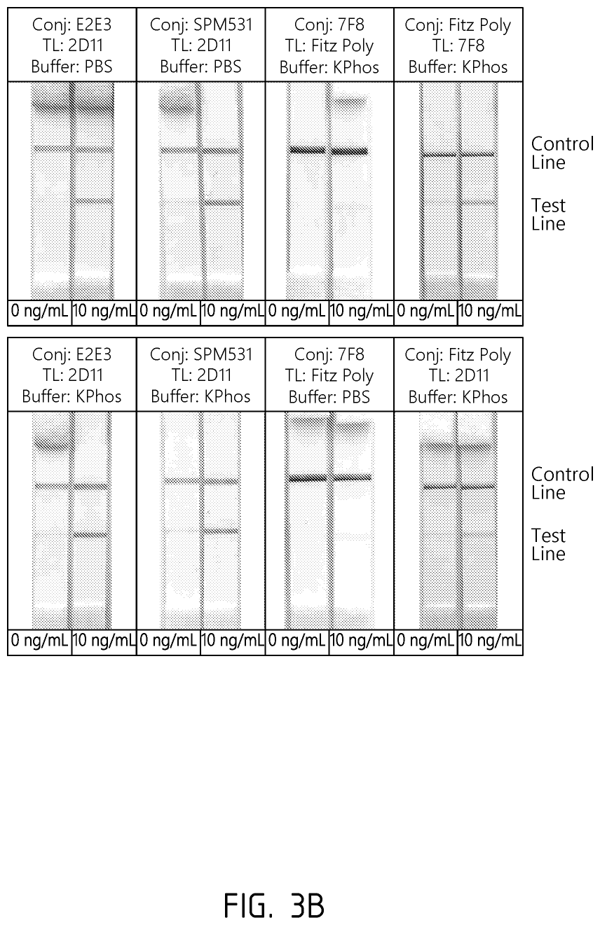

In this experiment (FIG. 3A), antibody pairing studies were performed to identify the best combination of antibodies for use in a direct (sandwich) lateral flow assay for equine insulin. For this experiment, equine insulin lateral flow assay test line intensities (millivolts) from insulin positive [10 ng/mL (288 uU/mL)] and insulin negative (0 ng/ml) equine plasma samples were compared, with different insulin antibody clones being utilized as the capture antibody and detector antibody, respectively. Top numbers for each pair of antibodies indicate “Ratio” which is the insulin positive sample lateral flow assay test line intensity divided by the insulin negative sample lateral flow assay test line intensity. Bottom numbers indicate “Difference” which is the difference between these same values. “Capture or Test Line Antibody” indicates the antibody striped on a nitrocellulose membrane at the lateral flow assay test line location, written as “vendor, clone number.” “Detector Antibody” indicates the antibody conjugated to a gold nanoparticle detector, also written as “vendor, clone number.” “KPhos Rxn Buffer” refers to assays that were conducted with the detector antibody having been conjugated to the gold nanoparticle detector in potassium phosphate reaction buffer, and “PBS Rxn Buffer” refers to assays that were conducted with the detector antibody having been conjugated to the gold nanoparticle detector in phosphate buffered saline reaction buffer. Ultimately, clone E2E3 was selected as the detector antibody and clone 2D11 was selected as the capture or test line antibody based upon the data presented, with the large ratios and differences resulting from use of the aforementioned antibodies appearing in bold text. The comparison table can be seen in FIG. 3A.

FIG. 3B illustrates a sample of images of equine insulin lateral flow assays described in FIG. 3A showing differences in test line intensities for insulin positive [10 ng/ml (288 uU/mL)] and insulin negative (0 ng/ml) equine plasma samples for: different combinations of insulin antibodies as the detector antibody (“Conj”) and capture or test line antibody (“TL”), respectively; and, with detector antibodies having been conjugated to the gold nanoparticle detector in either phosphate buffered saline (“PBS”) or sodium phosphate (“KPhos”) reaction buffer.

### Example 2. Detection Antibody Conjugation Protocols to Gold Nanoparticles and their Effects on Equine Insulin Lateral Flow Assays Correlation with Equine Insulin Radioimmunoassays

In a follow up study, experiments were conducted to identify the detection antibody conjugation protocol to gold nanoparticles that provided the strongest correlation between equine insulin LFAs and Cornell radioimmunoassays (RIAs). Table 1 displays: (1) equine plasma insulin concentration for 15 samples as determined by Cornell RIA and corresponding LFA test line intensity in millivolts (mV) resulting from various detection antibody (Mabtech E2E3) gold nanoparticle conjugation protocols, including (a) direct covalent conjugation to carboxylated gold nanoparticles in phosphate buffered saline (“PBS control”), (b) direct covalent conjugation to carboxylated gold nanoparticles in sodium phosphate (“NaPhos”), (c) direct covalent conjugation to carboxylated gold nanoparticles in polyethylene glycol and with a bovine serum albumin block (“TQD”), and (d) biotinylated antibody bound to streptavidin coated gold nanoparticles (“Bi-SA”); (2) correlation [coefficient of determination (“R²”) and Pearson correlation coefficient (“Pearson's r”)] of LFAs (test line millivolt reading) with Cornell RIAS (plasma insulin concentrations in uU/ml); and (3) confidence intervals of correlation data. (Note: Cornell RIAs performed by Animal Health Diagnostic Center, Cornell University College of Veterinary Medicine.)

TABLE 1

| Equine | Plasma Insulin | | | | |
|---|---|---|---|---|---|
| Plasma | Concentration | | | | |
| Sample | Cornell RIA Testing | PBS control | NaPhos | TQD | Bi-SA |
| ID | uU/mL | mV | mV | mV | mV |
| BS | 11.5 | 198 | 159 | 616 | 379 |
| FFF | 21.63 | 232 | 192 | 1695 | 483 |
| GG | 17.52 | 244 | 146 | 840 | 475 |
| BD | 70.56 | 690 | 1417 | 3394 | 1194 |
| EM | 54.04 | 302 | 283 | 629 | 1348 |
| AS | 68.67 | 725 | 770 | 2218 | 974 |
| DH | 157.29 | 2217 | 2715 | 4748 | 2562 |
| EO | 79.17 | 1833 | 2387 | 3211 | 1402 |
| ER | 108.25 | 4293 | 3169 | 5229 | 2160 |
| FK | 14.91 | 723 | 763 | 519 | 257 |
| AE | 135.48 | 2048 | 2879 | 3992 | 2039 |
| CZ | 165.35 | 2411 | 2622 | 4420 | 3056 |
| GL | 156.97 | 3182 | 3089 | 4828 | 2530 |
| JJJ | 182.25 | 2107 | 2897 | 5466 | 3111 |
| QQQ | 146.12 | 2945 | 2562 | 4800 | 3159 |

| | | | | |
|---|---|---|---|---|
| R² | 0.60 | 0.81 | 0.85 | 0.94 |
| Pearson's r | 0.77 | 0.90 | 0.92 | 0.97 |
| Confidence | (0.43, | (0.72, | (0.78, | (0.92, |
| Interval | 0.92) | 0.97) | 0.97) | 0.99) |

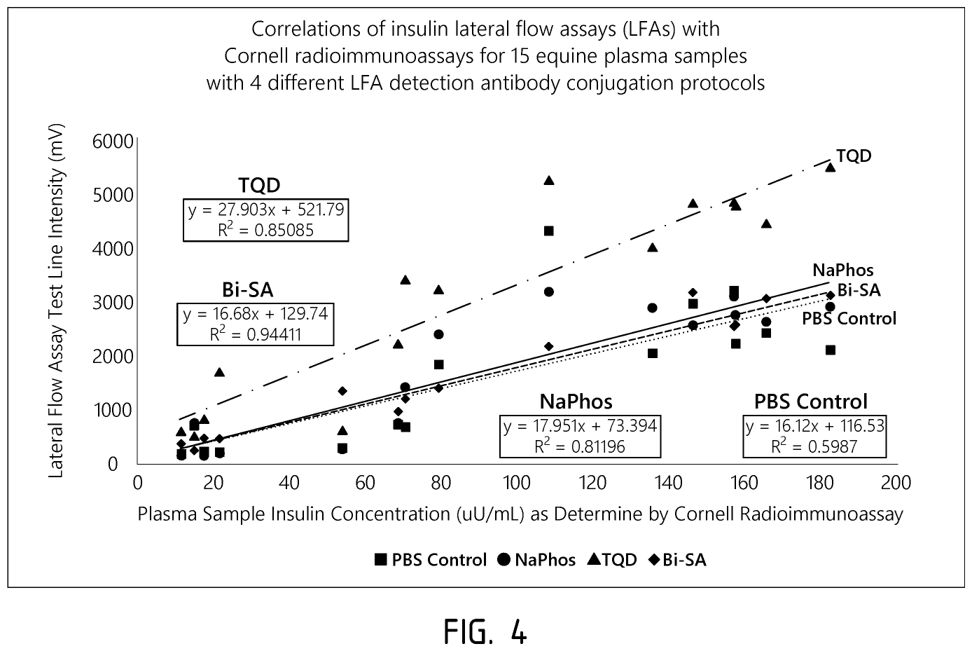

FIG. 4 displays the correlation of equine insulin LFAs with Cornell RIAs for a set of 15 equine plasma samples, with LFAs constructed from four different detection antibody conjugation protocols to gold nanoparticles. These experiments demonstrate that the detection antibody (Mabtech E2E3) conjugation protocol involving antibody biotinylation followed by binding of biotinylated antibody to streptavidin coated gold nanoparticles results in the strongest equine insulin LFA correlation with Cornell RIA and the highest confidence interval. Therefore, this detection antibody conjugation protocol was used in the final equine insulin lateral flow assay. Mabtech insulin antibody 2D11 was used as the capture or test line antibody in all assays.

### Example 3. Effects of Chase Buffer Formulation on Equine Insulin Lateral Flow Assay Correlation with Equine Plasma Insulin Concentration

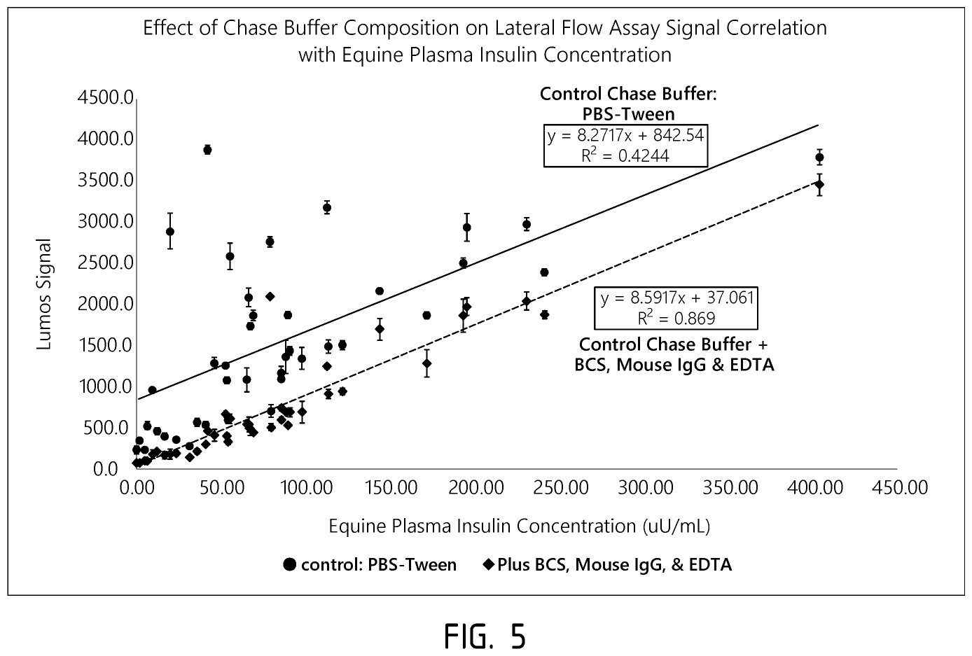

FIG. 5 illustrates the effect of two different chase buffer formulations on equine insulin lateral flow assay signal correlation with equine plasma insulin concentration. Before application to the lateral flow test strip, equine plasma samples were premixed with chase buffer consisting of either Ix phosphate buffered saline (PBS) with 1% Tween® by mass (10 mg/mL) or Ix PBS with 1% Tween plus 10% bovine calf serum (BCS) by mass, 50 μg/mL Mouse IgG and 15 mM EDTA. Addition of BCS, Mouse IgG and EDTA to the chase buffer significantly improved correlation of lateral flow assay test line signals (“Lumos Signal”) with equine plasma insulin concentrations. Therefore, this chase buffer composition was used in the final equine insulin lateral flow assay. Equine plasma insulin concentrations were determined by the Mercodia® Equine Insulin Elisa. (Note: error bars represent the standard deviation of the equine insulin lateral flow assay test line signals for each plasma sample at n=3.)

### Example 4. Whole Blood Filtering Capability of Various Blood Filter Pads and their Effects on Equine Insulin Lateral Flow Assay Sensitivity

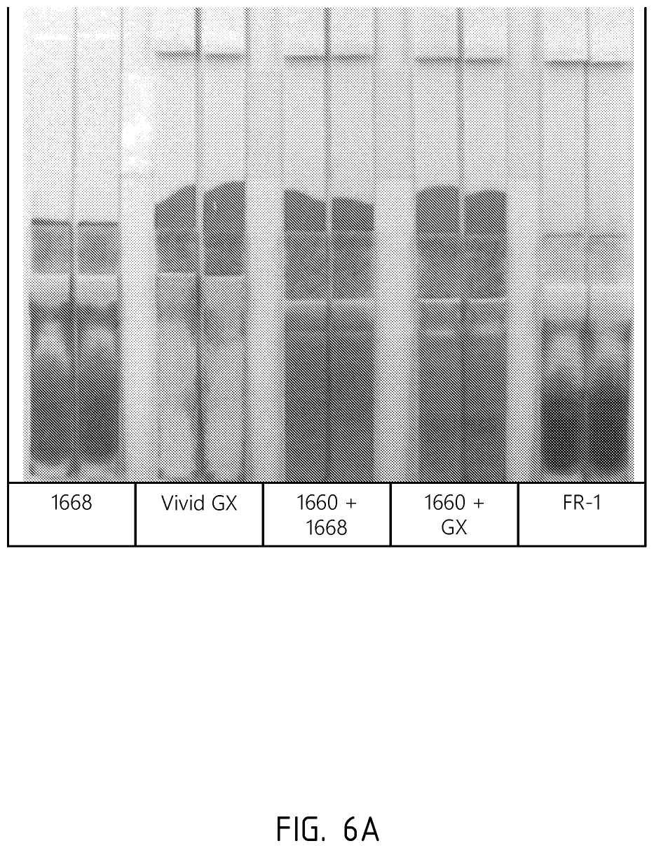

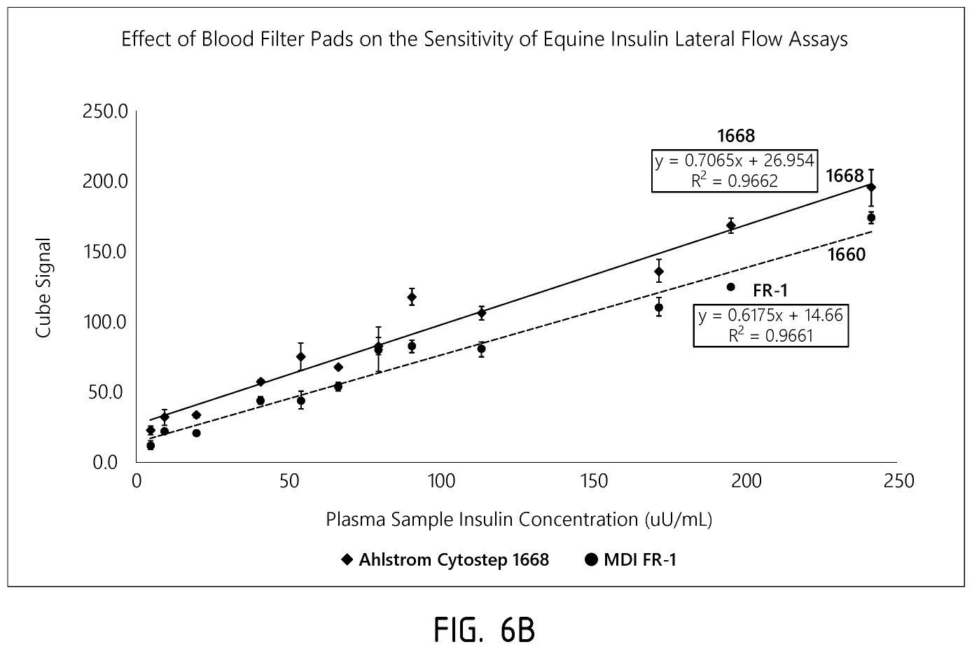

In this study, the whole blood filtering capability of various blood filter pads was investigated, along with the blood filter pads' effects on equine insulin lateral flow assay sensitivity. FIG. 6A illustrates images of equine insulin lateral flow assay test strips with different blood filter pads or combinations thereof to which an equine whole blood sample has been applied. The lateral flow assay strips with different blood filter pads or combinations thereof display varying degrees of blood migration onto the nitrocellulose membrane, which is undesirable due to the capability of blood on the nitrocellulose membrane to affect reading of the control and test line intensities by electronic readers. Blood filter pads 1668 and FR-1 each exhibited a capability to significantly reduce whole blood migration onto the nitrocellulose membrane in comparison with other membranes or combinations of membranes, and, therefore, these two blood filter pads were further analyzed for their relative effects on lateral flow assay sensitivity. [“1668”=Ahlstrom Cytostep®1668; “Vivid GX” or “GX”=Pall Vivid™ GX; “FR-1”, MDI Membrane Technologies FR-1] (Note: combinations of two blood filter pads are denoted by “+” symbol between the pads defined above.)

FIG. 6B illustrates a comparison of equine insulin lateral flow assay results using two different blood filter pads. “Cube Signal” is the test line intensity of lateral flow assays conducted on equine plasma samples as measured by a photographic electronic reader (Cube Reader, Chembio Diagnostic Systems, Inc.) with results reported in arbitrary units. Equine plasma sample insulin concentrations were determined by the Mercodia® Equine Insulin ELISA and are reported in microunits per milliliter (uU/mL). Correlation coefficients (coefficient of determination=“R²”) of cube signal readings with plasma insulin concentrations were similar for assays conducted with the Ahlstrom Cytostep® 1668 and the MDI Membrane Technologies FR-1 blood filter pads (0.9662 and 0.9661, respectively). However, the sensitivity of assays conducted with the Ahlstrom Cytostep® 1668 was greater than the sensitivity of assays performed with the MDI Membrane Technologies FR-1 as evidenced by the greater slope of the 1668 trendline (=0.7065) in comparison with the FR-1 trendline (=0.6175). Therefore, blood filter pad 1668 was selected for use in the final equine insulin lateral flow assay. (Note: error bars represent the standard deviation of the equine insulin lateral flow assay test line signals for each plasma sample at n=2.)

### Example 5. Demonstration of the Empirical Performance of a Direct (Sandwich) Lateral Flow Assay for Equine Insulin

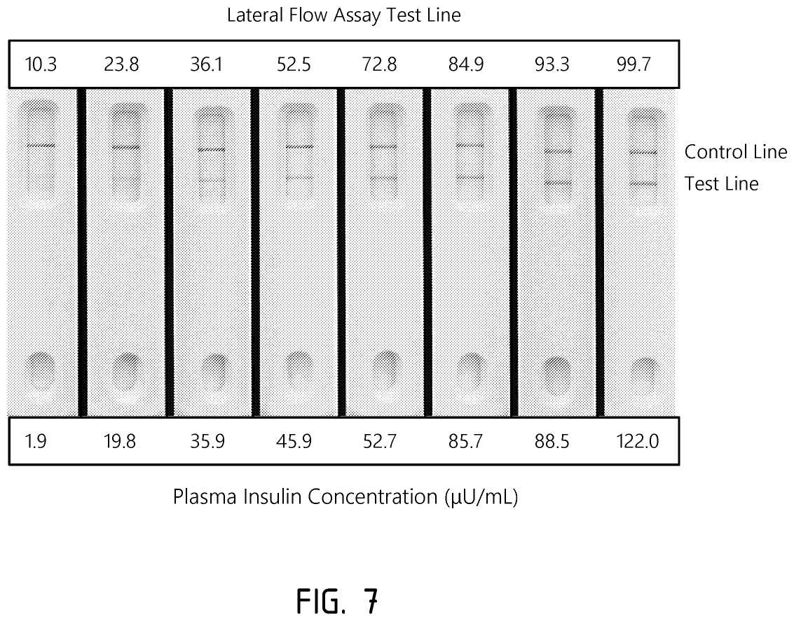

FIG. 7 illustrates an image of an equine insulin lateral flow assays displaying increasing test line intensity (left to right) with increasing concentration of insulin in equine plasma samples. Test line intensity (above) was measured with a photographic electronic reader (Cube Reader, Chembio Diagnostic Systems, Inc.) with results reported in arbitrary units. Equine plasma insulin concentrations (below) were previously determined by the Mercodia® Equine Insulin ELISA.

Although particular aspects are described herein, many variations and permutations of these aspects fall within the scope of the disclosure. Although some benefits and advantages are mentioned, the scope of the disclosure is not intended to be limited to particular benefits, uses, or objectives. Rather, aspects of the disclosure are intended to be broadly applicable to different detection technologies and device configurations some of which are illustrated by way of example in the figures and in the description.

What is claimed is:

1. A point of care testing method for equine insulin, the method comprising:
(a) extracting an equine whole blood sample from a horse;
(b) placing, at the point of care of the horse, the whole blood sample on a test strip of a lateral flow assay device, the test strip having
(i) a whole blood filter pad receiving the equine whole blood sample;
(ii) a conjugate pad carrying a conjugated insulin detection antibody, which is insulin antibody E2E3 biotinylated to streptavidin coated gold nanoparticles, the conjugated insulin detection antibody binding to equine insulin from the equine whole blood sample from the whole blood filter pad; and
(iii) a nitrocellulose membrane carrying insulin capture antibody 2D11, the insulin capture antibody binding to the conjugated insulin detection antibody bound to the equine insulin from the conjugate pad and forming a visible test line on the test strip;
(c) measuring, at the point of care of the horse, an intensity of the visible test line on the test strip with an optoelectronic reader carried by the lateral flow assay device, the optoelectronic reader being calibrated to determine an equine insulin concentration of the equine whole blood sample from the intensity of the visible test line on the test strip; and
(d) displaying, at the point of care of the horse, the equine insulin concentration of the equine whole blood sample measured by the optoelectronic reader.

2. The method of claim 1, wherein a volume of the equine whole blood sample is 10 μL to 1000 μL.

3. The method of claim 1, wherein the lateral flow assay device can determine the concentration of equine insulin at a concentration of 1 pg/mL to 100 pg/mL from an equine whole blood sample volume of 10 μL to 1000 μL.

4. The method of claim 1, wherein the lateral flow assay device can determine the concentration of equine insulin at a concentration of 10 ng/ml of the equine whole blood sample.

5. The method of claim 1, the equine whole blood sample includes a chase buffer including mouse IgG, EDTA, and bovine calf serum.

6. The method of claim 1, wherein the equine insulin concentration is displayed within three minutes from when the whole blood filter pad receives the equine whole blood sample.

7. A method comprising:
(a) determining a concentration of equine insulin in an equine whole blood sample using a lateral flow assay device, the lateral flow assay device having a test strip including
(i) a whole blood filter pad receiving the equine whole blood sample;
(ii) a conjugate pad carrying a conjugated insulin detection antibody, which is insulin antibody E2E3 biotinylated to streptavidin coated gold nanoparticles, the conjugated insulin detection antibody binding to the equine insulin from the equine whole blood sample; and
(iii) a nitrocellulose membrane carrying including insulin capture antibody 2D11, which binds to the conjugated insulin detection antibody bound to the equine insulin and forms a visible test line; and
(b) measuring an intensity of the visible test line, the intensity of the visible test line corresponding to the concentration of equine insulin in the equine whole blood sample.

8. The method of claim 7, wherein a volume of the equine whole blood sample is 10 μL to 1000 μL.

9. The method of claim 7, wherein the lateral flow assay device can determine the concentration of equine insulin at a concentration of 1 pg/mL to 100 pg/mL from an equine whole blood sample volume of 10 μL to 1000 μL.

10. The method of claim 7, wherein the lateral flow assay device can determine the concentration of equine insulin at a concentration of 10 ng/ml of the equine whole blood sample.

11. The method of claim 7, the equine whole blood sample includes a chase buffer including mouse IgG, EDTA, and bovine calf serum.

12. The method of claim 7, wherein the lateral flow assay device includes a calibrated optoelectronic reader that measures the intensity of the visible test line and displays the concentration of equine insulin.

13. The method of claim 7, wherein the concentration is determined within three minutes from when the whole blood filter pad receives the equine whole blood sample.

14. The method of claim 7, performed at a point of care of a horse.
